(19) United States
(12) Patent Application Publication
(10) Pub. No.: US 2002/0198150 A1
(43) Pub. Date: Dec. 26, 2002

(54) Methods of using colony stimulating factors in the treatment of tissue damage and ischemia

(76) Inventor: Ayelet Chajut, Ramat Hasharon (IL)

Correspondence Address: Cooper & Dunham LLP, 1185 Avenue of the Americas, New York, NY, 10036, US

(21) Appl. No.: 10/165,350

(22) Filed: Jun. 7, 2002

Related U.S. Application Data
(60) Provisional application No. 60/296,585, filed on Jun. 7, 2001.

(51) Int. Cl. A61K 38/18

(52) U.S. Cl. 514/12

(57) ABSTRACT

The present invention relates to novel uses of growth factors, particularly colony stimulating factors (CSFs), that stimulate migration and differentiation of stem cells in order to promote and enhance recovery from tissue trauma and ischemic events, including ischemia of the central nervous system, as well as for use in preventing or alleviating chronic degenerative processes, including neuronal degeneration

This application is a continuation-in-part and claims the benefit of U.S. Provisional Application No. 60/296,585, filed June 7, 2001, the contents of which are hereby incorporated by reference into this application.

### FIELD OF THE INVENTION

The present invention relates to novel uses of growth factors that stimulate migration and differentiation of stem cells, particularly colony stimulating factors (CSFs), in order to promote and enhance recovery from tissue trauma and ischemic events, including ischemia of the central nervous system, as well as for use in preventing or diminishing chronic degenerative changes.

### BACKGROUND OF THE INVENTION

Until recently, the fate of stem cells from an adult organism was thought to be restricted to their tissue origin. Stem cells of specific tissue origin have been known for quite some time to be capable of replenishing their corresponding damaged tissues, such as blood, muscle, liver, skin and brain.

However, several recent reports have shown that adult bone marrow cells injected into experimental animals, were able to migrate to different tissue types, differentiate to the corresponding cell type and contribute to healthy organ function:

(a) Bone marrow cells restored the liver architecture and its biochemical function in a mouse model of a lethal hereditary liver disease (E. Lagasse et al., Nature Med. 6, 1229 (2000))

(b) Bone marrow cells in mice were able to migrate into the brain and differentiate into phenotypic neuronal cells after or even without lethal irradiation of the mice (T. R. Brazelton et al., Science, 290, 1775 (2000); E. Mezey, et al., Science, 290, 1779 (2000)). Intravenously injected bone marrow stromal cells were also shown to enter the brain and to reduce neurological functional deficits after stroke in rats (J. Chen, et al., Stroke, 32, 1005 (2001)).

(c) Selected (lin⁻/c-kit⁺) hematopoietic stem cells (HSC) formed myocardium (myocytes, endothelial and smooth muscle cells) occupying 68% of the infarcted portion when injected into the contacting wall bordering myocardial infarcts (D. Orlic, et al., Nature, 410, 701 (2001)). Moreover, human bone-marrow-derived endothelial cell precursors injected intravenously after experimental myocardial infarction (MI), induced new blood vessel formation in the infarct-bed and proliferation of pre-existing vasculature (A. A. Kocher, et al., Nature Med. 7, 430 (2001)).

Protocols for increasing the levels of circulating HSC have been developed in the context of efforts to overcome the depletion of hematopoietic cells resulting from chemotherapy. Patients receiving chemotherapy may be treated with cytokines to stimulate expansion of HSC to overcome and prevent long lasting cytopenia.

Regimens known in the art to be capable of mobilizing increased numbers of bone-marrow-derived stem cells into the blood circulation include administration of colony-stimulating-factors (CSFs) or growth factors, sometimes in combination with chemotherapy. CSFs are increasingly used in the treatment of bone marrow transplant patients. Colony-stimulating factors are typically administered over several days or weeks. They may be injected intravenously or subcutaneously. The mobilized agent may be administered once daily for one to fourteen days. The first dose may be administered as early as immediately after the first diagnosis, or may begin after the final diagnosis (Bodine D M, et al., In vivo administration of stem cell factor to mice increases the absolute number of pluripotent hematopoietic stem cells, Blood 82: 445-455, 1993.)

The dose and the route of administration of the HSC-promoting agent may vary. R. Schots, et al. demonstrated that daily administration of 5 to 15 μg/kg of body weight of G-CSF for a total of 3 to 5 days is generally effective in inducing elevated levels of circulating HSC (R. Schots, et al., Bone Marrow Transplant. 17:509 (1996)). Moreover, more than one mobilization agent may be administered.

An additional use in the art of these stimulating agents is aimed at augmenting the stem cell content in the blood of potential stem cell donors prior to stem cell harvesting. Healthy donors are treated with CSFs, especially granulocyte colony-stimulating factor (G-CSF), leading to marrow stem cell release into the peripheral blood. The most common side effects are rash, mild to moderate bone pain, muscle pain, weakness, fever, headache and/or chills. The discomfort can usually be alleviated with analgesics such as paracetamol or non-steroid anti-inflammatory drugs.

Ischemia of the Brain

Brain injury such as trauma and stroke are among the leading causes of mortality and disability in the Western world.

Traumatic brain injury (TBI) is one of the most serious reasons for hospital admission and disability in modern society. Clinical experience suggests that TBI may be classified as primary damage occurring immediately after injury, and secondary damage that occurs during the several days following the injury. Current therapy of TBI is either surgical or else mainly symptomatic.

Cerebrovascular disease occurs predominately in the middle and late years of life. They cause approximately 200,000 deaths in the United States each year as well as considerable neurologic disability. The incidence of stroke increases with age and affects many elderly people, a rapidly growing segment of the population. These diseases cause ischemia, infarction and intracranial hemorrhage.

Stroke is an acute neurologic injury occurring as a result of interrupted blood supply, resulting in an insult to the brain. Most cerebrovascular diseases present as the abrupt onset of a focal neurological deficit. The deficit may remain fixed or it may either improve or progressively worsen, leading usually to irreversible neuronal damage at the core of the ischemic focus, whereas neuronal dysfunction in the penumbra may be reversible.

More prolonged periods of ischemia result in frank tissue necrosis. Cerebral edema follows and progresses over the subsequent 2 to 4 days. If the region of infarction is large, the edema may produce considerable mass effect with all of its attendant consequences. Damage to neuronal tissue can lead to severe disability and death. The extent of the damage is primarily affected by the location and extent of the injured tissue. Endogenous cascades activated in response to the acute insult play a role in the functional outcome. Efforts to minimize, limit and/or reverse the damage have the great potential of alleviating the clinical consequences.

Neuroprotective drugs are being developed in an effort to rescue neurons in the penumbra from dying although, as yet, none has been proven efficacious. One major problem with the proposed neuroprotective drugs is the very narrow therapeutic time window during which this type of therapy may be beneficial. It is generally considered that such agents must be administered within hours of the insult in order for them to prevent or diminish neuronal loss.

Recently, it has been disclosed that certain polypeptide growth factors may be used to treat central nervous system injuries (U.S. Pat. No. 6,214,796). This proposed method provides significant benefits because administration can occur a substantial amount of time following injury. The teachings of U.S. Pat. No. 6,214,796 include a vast list of candidate growth factors and neurotrophic factors, particularly certain fibroblast growth factors (FGFs). FGFs were previously known in the art to be involved in bone and cartilage remodeling and repair and as glia-activating factors. Patent application publication No. WO 96/34604 discusses methods of inhibition of intracellular acidification. Methods of attenuating acidification in a eukaryotic cell are provided as a means of inhibiting apoptosis (programmed cell death) in a cell, and alkalizing agents useful in the methods are disclosed.

Ischemia of the Heart

Myocardial infarction (MI) generally occurs when there is an abrupt decrease in coronary blood flow, usually following a thrombotic occlusion of a coronary artery previously narrowed by atherosclerosis.

It is one of the most common diagnoses in hospitalized patients in industrialized countries. In the United States, approximately 1.5 million myocardial infarctions occur each year. The mortality rate with acute infarction is approximately 30 percent. Although the mortality rate after admission for myocardial infarction has declined over the last two decades, approximately 1 of every 25 patients who survives the initial hospitalization dies in the first year after myocardial infarction. Survival is markedly reduced in elderly patients (over age 65), whose mortality rate is 20 percent at 1 month and 35 percent at 1 year after infarction.

In both cases, damage to cardiac tissue can lead to severe disability and death. The extent of the damage is primarily affected by the location and extent of the injured tissue. Endogenous cascades activated in response to the acute insult play a role in the functional outcome. Efforts to minimize, limit and/or reverse the damage have the great potential of alleviating the clinical consequences.

### SUMMARY OF THE INVENTION

The present invention relates to novel uses of growth factors that stimulate migration and differentiation of stem cells, particularly colony stimulating factors (CSFs), in order to promote and enhance recovery from tissue trauma and ischemic events, including ischemia of the central nervous system, as well as for use in preventing or diminishing chronic degenerative changes. The present invention further provides methods for alleviating or reducing symptoms and signs associated with damaged neuronal tissues, whether resulting from tissue trauma or from chronic or acute degenerative changes, and for promoting or enhancing recovery in a patient who has suffered an injury to the central nervous system, the method comprising administering to the patient a pharmaceutical composition comprising at least one colony stimulating growth factor in sufficient dosage to increase the number of bone-marrow derived stem cells in the circulation of said patient.

### DETAILED DESCRIPTION

The present invention provides methods for therapeutic improvement of the symptoms and signs associated with damaged tissues, whether resulting from tissue trauma, or from chronic degenerative changes. It is a further objective of the present invention to provide methods leading to functional improvement after traumatic ischemic events, including but not limited to, MI, traumatic brain injury (TBI) or cerebral stroke, by affecting reperfusion and regeneration of the ischemic tissue.

The present invention provides pharmaceutical compositions to reduce or even prevent tissue damage or degeneration due to acute injury to the CNS as described, or due to other insults, such as chronic hepatic disease or renal failure.

The compositions of the present invention may also be effective in treating certain chronic degenerative diseases that are characterized by gradual selective neuronal loss. In this connection, the compositions of the present invention are contemplated as therapeutically effective in the treatment of Parkinson's disease, Alzheimer's disease, epilepsy, depression, ALS (Amyotrophic lateral sclerosis), Huntington's disease and any other disease-induced dementia (such as HIV-induced dementia, for example).

These effects will be achieved by administering an agent that stimulates the mobilization of bone marrow-derived stem cells into the bloodstream. Representative agents useful in the methods of the invention include, for example, granulocyte-colony stimulating factor (G-CSF), granulocyte-macrophage-colony stimulating factor (GM-CSF), stem cell factor (SCF), interleukin-3 (IL-3) and interleukin-6 (IL-6). These factors have all been shown to be capable of mobilizing bone marrow-derived stem cells (T. J. Hoffmann, et al., Exp. Hematol. 22, 1016 (1994); T. de Revel, et al., Blood, 83:3795 (1994); R. Schots, et al., Bone Marrow Transplant. 17:509 (1996)).

According to the present disclosure in vivo treatment with a growth factor capable of stimulating or increasing the number of bone marrow-derived stem cells in the circulation is beneficial for diseases and conditions requiring tissue regeneration or for preventing or ameliorating tissue degeneration, in tissues other than the hematopoietic system, or bone and cartilage.

According to a currently preferred embodiment of the invention these objectives are accomplished by treating an individual with one of the growth factors known as colony stimulating factors.

In a more preferred embodiment of the invention, in vivo treatment of an individual is performed using a colony stimulating factor selected from the group consisting of granulocyte-colony stimulating factor (G-CSF), granulocyte-macrophage-colony stimulating factor (GM-CSF), stem cell factor (SCF), interleukin-3 (IL-3) and interleukin-6 (IL-6).

According to a yet more preferred embodiment of the invention, the growth factor used for treatment of tissue trauma and ischemic insults is G-CSF.

One embodiment according to the invention provides for use of a colony stimulating factor for the preparation of a medicament for the treatment of tissue trauma or ischemia.

Another embodiment according to the invention is a pharmaceutical composition for the treatment of ischemia or tissue trauma comprising as an active ingredient a colony stimulating factor.

Yet another embodiment according to the current invention provides a method for the treatment of an individual in need thereof with a composition comprising as an active ingredient a therapeutically effective amount of a colony stimulating factor, whereby the treatment decreases the damage resulting from ischemic or hypoxic insults.

Yet another embodiment according to the current invention provides a method for the treatment of an individual in need thereof with a composition comprising as an active ingredient a therapeutically effective amount of a colony stimulating factor, whereby the treatment enhances or promotes the regeneration of a tissue other than a hematopoietic tissue, bone or cartilage.

Yet another embodiment according to the current invention provides a method for the treatment of an individual in need thereof with a composition comprising as an active ingredient a therapeutically effective amount of a colony stimulating factor, preferably G-CSF, whereby the treatment prevents degeneration of a tissue other than a hematopoietic tissue, bone or cartilage.

Also included in the invention are “functional polypeptide growth factors,” which possess one or more of the biological functions or activities of the colony stimulating factors described herein. These functions or activities are described in detail herein and concern, primarily, increasing the number of bone marrow-derived stem cells in the circulation of the individual receiving the treatment and enhancement of recovery following an ischemic event.

Accordingly, alternate molecular forms of polypeptide growth factors are within the scope of the invention. Alternatively, polypeptide growth factors useful in the invention can consist of active fragments of the factors. By “active fragment,” as used herein in reference to polypeptide growth factors, is meant any portion of a polypeptide that is capable of invoking the same activity as the full-length polypeptide. The active fragment will produce at least 40%, preferably at least 50%, more preferably at least 70%, and most preferably at least 90% (including up to 100%) of the activity of the full-length polypeptide. The activity of any given fragment can be readily determined in any number of ways. For example, a fragment of G-CSF that, when administered according to the methods of the invention described herein, is shown to perform in functional tests in a manner comparable to the performance that is produced by administration of the full-length G-CSF polypeptide, would be an “active fragment” of G-CSF. It is well within the abilities of the skilled artisan to determine whether a polypeptide growth factor, regardless of size, retains the functional activity of a full length, wild type polypeptide growth factor.

The invention also comprehends that homologous polypeptides, which possess one or more of the biological functions or activities of the colony stimulating factors described herein, can be used in the same fashion as the herein or aforementioned polypeptides. By homologous polypeptides is meant isolated and/or purified polypeptides having at least about 70%, preferably at least about 75%, more preferably at least about 80%, even more preferably at least about 90%, most preferably at least about 95% homology to a colony stimulating factor, or to a functional polypeptide growth factor described above.

As used herein, both “protein” and “polypeptide” mean any chain of amino acid residues, regardless of length or post-translational modification (e.g., glycosylation or phosphorylation). The polypeptide growth factors useful in the invention are referred to as “substantially pure,” meaning that a composition containing the polypeptide is at least 60% by weight (dry weight) the polypeptide of interest, e.g., a G-CSF polypeptide. Preferably, the polypeptide composition is at least 75%, more preferably at least 90%, most preferably at least 99%, by weight, the polypeptide of interest. Purity can be measured by any appropriate standard method, e.g., column chromatography, polyacrylamide gel electrophoresis, or HPLC analysis.

The polypeptide growth factors useful in the invention can be naturally occurring, synthetic, or recombinant molecules consisting of a hybrid or chimeric polypeptide with one portion, for example, being G-CSF and a second portion being a distinct polypeptide. These factors can be purified from a biological sample, chemically synthesized, or produced recombinantly by standard techniques (see e.g., Ausubel et al., Current Protocols in Molecular Biology, New York, John Wiley and Sons, 1993; Pouwels et al., Cloning Vectors: A Laboratory Manual, 1985, Supp. 1987). CSFs in general, and G-CSF in particular, can be prepared as described in U.S. Pat. No. 5,849,883 and/or PCT Publication No. WO 92/14480 Al. Additionally, NEUPOGEN®, also known as Filgrastim, is a recombinant human granulocyte colony-stimulating factor (G-CSF) which is a commercially available approved drug (Amgen, Thousand Oaks, Calif., USA).

The treatment regimen according to the invention is carried out, in terms of administration mode, timing of the administration, and dosage, so that the functional recovery of the patient from the adverse consequences of the ischemic event or central nervous system injury is improved, i.e., the patient's motor skills (e.g., posture, balance, grasp, or gait), cognitive skills, speech, and/or sensory perception (including visual ability, taste, olfaction, and proprioception) improve as a result of polypeptide growth factor administration according to the invention.

The invention can be used to treat the adverse consequences of central nervous system injuries that result from any of a variety of conditions. Preferably, the invention can be used to treat an ischemic episode, more preferably a cerebral ischemic episode. A cerebral ischemic episode can be caused by a condition selected from the group comprising thrombus, embolus, systemic hypotension, hypertension, hypertensive cerebral vascular disease, rupture of an aneurysm, angioma, blood dyscrasias, cardiac failure, cardiac arrest, cardiogenic shock, septic shock, head trauma, spinal cord trauma, seizure, bleeding from a tumor, traumatic brain injury, spinal injury and other blood loss.

Where the ischemia is associated with a stroke, it can be either global or focal ischemia, as defined below. It is believed that the administration of polypeptide growth factors according to the invention is effective, even though administration occurs a significant amount of time following the injury, at least in part because these polypeptides stimulate the growth of new processes from neurons. In addition, polypeptide growth factors may protect against retrograde neuronal death, i.e., death of the neurons that formed synapses with those that died in the area of the infarct. By “ischemic episode” or “ischemic event” is meant any circumstance that results in a deficient supply of blood to a tissue. “Stroke” is defined as a cerebral ischemic episode or event that results from a deficiency in the blood supply to the brain. A cerebral ischemic episode or event can be a global ischemic event or a focal ischemic event. The spinal cord, which is also a part of the central nervous system, is equally susceptible to ischemia resulting from diminished blood flow. An ischemic episode may be caused by a constriction or obstruction of a blood vessel, as occurs in the case of a thrombus or embolus. An ischemic episode or event, preferably a cerebral ischemic episode or event, may be caused by hypertension, hypertensive cerebral vascular disease, rupture of an aneurysm, a constriction or obstruction of a blood vessel as occurs in the case of a thrombus or embolus, angioma, blood dyscrasias, any form of compromised cardiac function including cardiac arrest or failure, systemic hypotension, cardiogenic shock, septic shock, spinal cord trauma, head trauma, seizure, bleeding from a tumor, or other blood loss.

It is expected that the invention will also be useful for treating injuries to the central nervous system that are caused by mechanical force, such as a blow to the head or spine. Trauma can involve a tissue insult such as an abrasion, incision, contusion, puncture, compression, etc., such as can arise from traumatic contact of a foreign object with any locus of or appurtenant to the head, neck, or vertebral column. Other forms of traumatic injury can arise from constriction or compression of CNS tissue by an inappropriate accumulation of fluid (e.g., a blockade or dysfunction of normal cerebrospinal fluid or vitreous humor fluid production, turnover, or volume regulation, or a subdural or intracranial hematoma or edema). Similarly, traumatic constriction or compression can arise from the presence of a mass of abnormal tissue, such as a metastatic or primary tumor.

By “focal ischemia”, as used herein in reference to the central nervous system, is meant the condition that results from the blockage of a single artery that supplies blood to the brain or spinal cord, resulting in the death of all cellular elements (pan-necrosis) in the territory supplied by that artery.

By “global ischemia”, as used herein in reference to the central nervous system, is meant the condition that results from a general diminution of blood flow to the entire brain, forebrain, or spinal cord, which causes the death of neurons in selectively vulnerable regions throughout these tissues. The pathology in each of these cases is quite different, as are the clinical correlates. Models of focal ischemia apply to patients with focal cerebral infarction, while models of global ischemia are analogous to cardiac arrest, and other causes of systemic hypotension.

The term “neurotoxic stress” as used herein is intended to comprehend any stress that is toxic to normal neural cells (and may cause their death or apoptosis). Such stress may be oxidative stress (hypoxia or hyperoxia) or ischemia or trauma, and/or it may involve subjecting the cells to a substance that is toxic to the cells in vivo, such as glutamate or dopamine or the A protein, or any substance or treatment that causes oxidative stress. The neurotoxic substance may be endogenous or exogenous, and the term neurotoxic is also intended to comprehend exposure to various known neurotoxins, including organophosphorous poisoning, or any other insult of this type.

The method of the invention has several advantages. First, polypeptide growth factors can be administered hours, days, weeks, or even months following an injury to the central nervous system. This is advantageous because there is no way to anticipate when such an injury will occur. All of the events that cause ischemia or trauma, as discussed above, are unpredictable. Second, this therapeutic regimen improves functional performance without adverse side effects.

The treatment regimen according to the invention is carried out, in terms of administration mode, timing of the administration, and dosage, so that the functional recovery of the patient from the adverse consequences of the central nervous system injury is improved, i.e., the patient's motor skills (e.g., posture, balance, grasp, or gait), cognitive skills, speech, and/or sensory perception (including visual ability, taste, olfaction, and proprioception) improve as a result of polypeptide growth factor administration according to the invention.

The present invention discloses for the first time the utility of growth factors capable of recruiting or mobilizing stem cells, including colony-stimulating factors (CSFs) in general, and G-CSF treatment in particular, for improving clinical and functional outcome after tissue trauma and for inducing organ regeneration in animals, including humans. These beneficial properties of CSFs are achieved by affecting bone marrow-derived stem cell mobilization by in vivo administration of the growth factor, thereby providing elevated numbers of stem cells in the circulation.

This approach has several distinct advantages over any hitherto available or suggested therapy, including:

Greater safety—Induction of augmentation and mobilization of autologous-bone marrow-derived stem cells by CSFs in general and G-CSF in particular is a known and experienced manipulation that has been generally well tolerated, with no apparent dose-limiting toxicity and no serious side effects. The use of autologous bone marrow-derived stem cells is not accompanied by any necessity for immunosuppressive medication as is needed after organ or allogeneic bone marrow transplantation.

A longer therapeutic window while preserving the favorable or beneficial effects

Preserving the endogenous potential of cell differentiation to the specific cells required, as bone marrow-derived stem cells have the potential of transactivation to various cell lines

Pharmacology

The compositions for these novel uses contain, in addition to the active ingredient, conventional pharmaceutically acceptable carriers, diluents and the like. Liquid forms may be prepared for oral administration or for injection, the term injection including subcutaneous, transdermal, intravenous, intramuscular, intrathecal, and other parenteral routes of administration. The liquid compositions include aqueous solutions, with or without organic co-solvents, aqueous or oil suspensions, emulsions with edible oils, as well as similar pharmaceutical vehicles. In addition, the compositions for use in the novel treatments of the present invention may be formed as aerosols, for intranasal and like administration.

The active dose for humans is generally in the range of from 0.5 μg/kg to about 1,000 μg/kg of body weight, preferably 1 to 50 μg/kg body weight, most preferably 5 to 15 μg per kg of body weight in a regimen where administration is 1-4 times/day preferably once or twice daily for a total of 1 to 14 days, preferably 3 to 5 days. However, administration every two days may also be possible, as the drug has rather prolonged action. Typically, the polypeptide growth factors are administered intravenously at concentrations ranging from 1-100 μg/kg/hour. However, it is evident to one skilled in the art that dosages would be determined by the attending physician, according to the disease to be treated, method of administration, patient's age, weight, contraindications and the like.

Preferably the CSF should not be given immediately after the injury or ischemic event. Without being bound by theory, this is to avoid increasing the inflammatory reaction. Treatment may commence within about one month after the injury or ischemic event, preferably on any one of days 1-30 most preferably on any one of days 1-7 after the injury or ischemic event.

The compounds are administered for the above-defined novel uses in conventional pharmaceutical forms, with the required solvents, diluents, excipients, etc. to produce a physiologically acceptable formulation. They can be administered by any of the conventional routes of administration.

It will be appreciated that the most appropriate administration of the pharmaceutical compositions of the present invention will depend on the type of injury or disease being treated. Thus, the treatment of an acute event will necessitate systemic administration of the drug as rapidly as possible after induction of the injury. On the other hand, diminution of chronic degenerative damage will necessitate a sustained dosage regimen.

Experimental Models:

CNS Injury

The potential of bone marrow stem cell-mediated therapy induced by colony stimulating factor (CSF) treatment for treating CNS injury is evaluated in animal models. The models represent varying levels of complexity, and evaluation is performed by comparing control animals to agent-treated animals. The efficacy of such treatments is evaluated in terms of clinical outcome, neurological deficit, dose-response and therapeutic window. Test animals are treated with a cytokine prepared in suitable buffer. Control animals are treated with buffer only.

1. Closed Head injury (CHI)—Experimental TBI produces a series of events contributing to neurological and neurometabolic cascades, which are related to the degree and extent of behavioral deficits. CHI is induced under anesthesia, while a weight is allowed a free fall from a prefixed height over the exposed skull covering the left hemisphere in the midcoronal plane (Chen et al, J. Neurotrauma 13:557 (1996)).2. Transient middle cerebral artery occlusion (MCAO)—A 90 to 120 minute transient focal ischemia is performed in adult, male Sprague Dawley rats, weighing 300-370 grams. The method employed is the intraluminal suture MCAO (Longa et al., Stroke 30:84 (1989); Dogan et al., J. Neurochem. 72:765 (1999)). Briefly, under halothane anesthesia, a 3-0 nylon suture material coated with Poly-L-Lysine is inserted into the right internal carotid artery (ICA) through a hole in the external carotid artery (ECA). The nylon thread is pushed into the ICA to the right middle cerebral artery (MCA) origin (20-23 mm). 90-120 minutes later the thread is pulled out, the animal is closed and allowed to recover.

3. Permanent middle cerebral artery occlusion (MCAO)—Occlusion is permanent and unilateral, and is induced by electrocoagulation of MCA. Both this method and method #2 above lead to focal brain ischemia at the ipsilateral side of the brain cortex, leaving the contralateral side intact (control).

Evaluation Process:

The efficacy of the cytokine, preferably G-CSF, is determined by mortality rate, weight gain, infarct volume and by short and long term clinical and neurophysiological outcome in surviving animals. Infarct volumes are assessed histologically (R. A. Knight et al., Stroke 25:1252 (1994); J. Mintorovitch et al., Magn. Reson. Med. 18:39 (1991)).

The staircase test (C. P. Montoya et al., J Neurosci Methods 36:219 (1991)) or the motor disability scale according to Bederson's method (J. B. Bederson et al., Stroke 17:472 (1986)) is employed to evaluate functional outcome following MCAO. The animals are followed for different time intervals. At each time point (24 h, 1 week, 3 weeks, 6 weeks and 8 weeks) animals are sacrificed, and cardiac perfusion with 4% formaldehyde in PBS is performed. Brains are removed and coronal sections are prepared for processing and paraffin embedding. They are then stained with TCC. The infarct area is measured in these sections using computerized image analysis.

Validation of the colony stimulating factor treatment on the above animal models provides new avenues for treatment of human brain injury.

Myocardial Infarction:

The potential of bone marrow stem cell-mediated therapy induced by CSF treatment as a tool for treating myocardial infarction is evaluated in animal models of varying levels of complexity (A. A. Kocher, et al., Nature Med. 7:430 (2001); Q. Li et al., J. Clin. Invest. 100:1991 (1997)), by comparison of control animals to agent-treated animals. The efficacy of such treatments is evaluated both in terms of clinical outcome, especially functional recovery, and in terms of dose-response and therapeutic window. The dosage tested is as described above for the models of CNS injury. See Itescu, PCT Patent Application, International Publication Number WO 01/94420 A1.

The preferred methods, materials, and examples that will now be described are illustrative only and are not intended to be limiting; materials and methods similar or equivalent to those described herein can be used in practice or testing of the invention. Other features and advantages of the invention will be apparent from the above detailed description, and from the claims.

All publications, patents, patent applications, and other references cited herein are incorporated by reference in their entirety.

### EXAMPLE 1

G-CSF-Induced Colony Formation

The ability of G-CSF to induce colony formation was assessed, as reported in the literature [Bodine D M, et.al., (1993). “—In vivo administration of stem cell factor to mice increases the absolute number of pluripotent hematopoietic stem cells.” Blood 82(2): 445-55; Bodine D. M. et. al., (1994). “—Efficient retrovirus transduction of mouse pluripotent hematopoietic stem cells mobilized into the peripheral blood by treatment with granulocyte colony-stimulating factor and stem cell factor.” Blood 84(5): 1482-91]

Murine G-CSF (Peprotech cat. # 250-05), in 0.9% NaCl, pH 4.55, 5% fetal calf serum (FCS), was administered to experimental animals. Four animal groups (2 i 5 experimental and 2 control groups) were assessed, with three animals comprising each group. Injections for experimental and control animals were performed as follows:

1. G-CSF injected daily for three days (experimental)

2. Solvent injected daily for three days (control)

3. G-CSF injected daily for five days (experimental)

4. Solvent injected daily for five days (control)

Preparation of G-CSF Solution and Injection Thereof

Murine G-CSF (Peprotech cat# 250-05) was reconstituted in double distilled water (DDW) to a concentration of 2 mg/ml and then diluted in solvent solution (0.9% NaCl, pH 4.55, 5% FCS) to a concentration of 0.5 mg/ml (stock solution). Aliquots were frozen at −20° C. and were then thawed and diluted immediately before use. The stock solution was diluted 1/30 in the solvent to a final concentration of 16.6 μg/ml.

200 μg/kg/day G-CSF was injected sub-cutaneously once daily for three or five days, as per the schedule presented above, in a final volume of 250 μl.

Collection of Peripheral Blood

Six hours after the final G-CSF injection, 0.5-0.7 μl blood were collected with a syringe from the heart of anaesthetized mice and added to a heparin-PBS solution (5000U/ml, Sigma, cat. #H-3149, final volume of 50 μl)

Quantification of Progenitor Content

Total mononuclear cells (MNC) and colony formation were quantified by standard methods as described briefly below.

Evaluation

Each colony that subsequently developed was the result of the proliferation of a single progenitor cell. In normal peripheral blood, there are 1-5 progenitor cells per 20 μl blood. After mobilization, there are 50-100 progenitor cells per 20 μl blood.

Results

Quantification of progenitor content:

Method #1

10 and 20 l of total blood were plated onto 1 ml methyl cellulose (from both day 3 and day 5 samples). Colonies were counted with a microscope after 7 days of colony growth. Control group, injected with solvent solution: 3 mice; experimental group, injected with G-CSF solution: 3 mice.

Day 3:

The total WBC count (counted in 1% acetic acid, 0.1% crystal violet in DDW on collected blood) was as follows:

| Control group: | G-CSF group: |
|---|---|
| 1) 6600 cells/l | 4) 18000 cells/l |
| 2) 1000 cells/l | 5) 13270 cells/l |
| 3) 3066 cells/l | 6) 8900 cells/l |
| Average: 3555 cells/l | Average: 13400 cells/l |

Colony count:

| | | | | | | | Average/ |
|---|---|---|---|---|---|---|---|
| | μl blood | | CFU- | CFU- | | total/20 | 20 μl |
| Mouse | plated | BFU-E | GM | GEMM | total | μl blood | blood |
| control | | | | | | | |
| 1 | 10 | 3 | 3 | 0 | 6 | 9 | 16.0 |
| | 20 | 2 | 4 | 0 | 6 | | |
| 2 | 10 | 0 | 14 | 0 | 14 | 19 | |
| | 20 | 2 | 8 | 0 | 10 | | |
| 3 | 10 | 15 | 4 | 0 | 19 | 20 | |
| | 20 | 0 | 1 | 0 | 1 | | |
| +G-CSF | | | | | | | |
| 4 | 10 | 0 | 2 | 0 | 2 | 4 | 7.3 |
| | 20 | 1 | 3 | 0 | 4 | | |
| 5 | 10 | 2 | 2 | 0 | 4 | 11 | |
| | 20 | 3 | 8 | 3 | 14 | | |
| 6 | 10 | 1 | 3 | 0 | 4 | 7 | |
| | 20 | 0 | 4 | 1 | 5 | | |

Day 5:

The total WBC count (counted in 1% acetic acid, 0.1% crystal violet in DDW in collected blood):

| Control group: | G-CSF group: |
|---|---|
| 7) 4800 cells/l | 10) 9940 cells/l |
| 8) 4270 cells/l | 11) 9470 cells/l |
| 9) 3870 cells/l | 12) 6773 cells/l |
| Average: 4313 cells/l | Average: 8714 cells/l |

Colony count:

| | | | | | | | Average/ |
|---|---|---|---|---|---|---|---|
| | μl blood | | CFU- | CFU- | | total/20 | 20 μl |
| Mouse | plated | BFU-E | GM | GEMM | total | μl blood | blood |
| control | | | | | | | |
| 7 | 10 | 5 | 4 | 0 | 9 | 14 | 8.8 |
| | 20 | 2 | 8 | 0 | 10 | | |
| 8 | 10 | 2 | 4 | 0 | 6 | 6 | |
| | 20 | 0 | 0 | 0 | 0 | | |
| 9 | 10 | 0 | 1 | 0 | 1 | 6.5 | |
| | 20 | 3 | 7 | 1 | 11 | | |
| +G-CSF | | | | | | | |
| 10 | 10 | 1 | 8 | 1 | 10 | 25 | 22.2 |
| | 20 | 12 | 14 | 4 | 30 | | |
| 11 | 10 | 11 | 9 | 1 | 21 | 26 | |
| | 20 | 5 | 4 | 1 | 10 | | |
| 12 | 10 | 2 | 2 | 0 | 4 | 15.5 | |
| | 20 | 9 | 14 | 0 | 23 | | |

Method #2: Separation of MNC on Ficoll gradient (performed only for the sample collected on day 5). Cells were counted in 1% acetic acid, 0.1% crystal violet in DDW. 2×10⁵ and 5×10⁵ WBC were each plated onto 1 ml methylcellulose. Colonies were counted after 7 days. Note that the total WBC number was determined (in 1% acetic acid, 0.1% crystal violet in DDW) in order to enable plating of the desired number of MNC on methylcellulose:

| Control group: | G-CSF group: |
|---|---|
| 7) 4760 cells/l | 10) 6500 cells/l |
| 8) 5050 cells/l | 11) 8550 cells/l |
| 9) 3706 cells/l | 12) 5500 cells/l |

Colony count:

| | | | | | | | average |
|---|---|---|---|---|---|---|---|
| | number | | | | | colonies/ | colonies/ |
| | of MNC | BFU- | CFU- | CFU- | | 1 × 10⁵ | 1 × 10⁵ |
| Mouse | plated | E | GM | GEMM | total | MNC | MNC |
| control | | | | | | | |
| 7 | 2 × 10⁵ | 3 | 12 | 6 | 21 | 11 | 3.8 |
| | 5 × 10⁵ | 11 | 23 | 3 | 37 | 7 | |
| 8 | 2 × 10⁵ | 0 | 0 | 0 | 0 | 0 | |
| | 5 × 10⁵ | 3 | 12 | 1 | 16 | 3 | |
| 9 | 2 × 10⁵ | 0 | 0 | 0 | 0 | 0 | |
| | 5 × 10⁵ | 2 | 7 | 1 | 10 | 2 | |
| +G-CSF | | | | | | | |
| 10 | 2 × 10⁵ | 11 | 17 | 19 | 37 | 19 | 36 |
| | 5 × 10⁵ | 32 | 50 | 17 | 99 | 20 | |
| 11 | 2 × 10⁵ | 14 | 20 | 5 | 39 | 20 | |
| | 5 × 10⁵ | 19 | 27 | 3 | 49 | 10 | |
| 12 | 2 × 10⁵ | 54 | 69 | 13 | 136 | 68 | |
| | 5 × 10⁵ | 220 | 148 | 40 | 408 | 82 | |

The maximal effect due to injected G-CSF was observed for the sample collected on day 5, following 5 consecutive IP injections of 200 μg/kg/day each.

### EXAMPLE 2

Experimental Design for Determination of the Effect of G-CSF-Induced Progenitor Cell Mobilization on MCAO-Induced Brain Damage

The experiment is divided into two parts:

I. G-CSF is administered for 5 consecutive days at a dose of 200 μg/kg/day, whereby the last administration is the same day as performance of the MCAO.

II. G-CSF is administered for 5 consecutive days at a dose of 200 μg/kg/day, whereby the first administration is 24 hours following performance of the MCAO.

For both parts of the experiment, performance and behavioral analysis (Montoya neurological motor analysis) of the animal after carrying out a permanent MCAO are determined:

1. Permanent MCAO

Permanent MCAO is accomplished by micropolar coagulation of the MCA. Briefly, anesthesia is induced by Equithesine i.p. (3-4 ml/kg). The left MCA is exposed using a subtemporal approach, leaving the zygomatic arch intact. The animals are placed in lateral recumbency and a 1-cm vertical skin incision is made between the left orbit and the external auditory canal. The underlying fascia are removed and the exposed temporalis muscle bluntly dissected and retracted to expose the inferior part of the temporal fossa. A small craniectomy is performed using a dental drill at the junction between the medial wall and the roof of the temporal fossa, approximately 0.5 mm dorsal to the foramen ovale. The dura mater is removed, and the main truck of the MCA is exposed proximal to the olfactory tract and occluded by micropolar coagulation (Tamura A. et al., J Cereb Blood Flow Metab. 1:53-60 (1981)). The occluded MCA is severed to prevent recanalisation. The muscle and skin are sutured using 3/0 or 4/0 Silk. The blood-flow before and after the occlusion is measured by Doppler. The experiment is considered successful when the remaining flow is −10% of the blood flow before the occlusion. In addition, upon recovery from the operation and anesthesia the mice are checked for paresis as an additional indication of brain damage.

2. Behavioral Test

The behavioral tests designed to assess the separate motor ability of each brain hemisphere of the model animal are instrumental in obtaining a primary indication of brain damage. Amongst the different behavioral and neurological tests the most accepted is that which utilizes the staircase apparatus, as developed by Montoya in C. P. Montoya et al., J Neurosci Methods 36, 219 (1991).

The staircase apparatus provides a simple and easy-to-quantify measure of skilled paw reaching in both rat and mice. The design allows separate measurements of reaching capacity with the left and the right paws. The test is sensitive to unilateral lesions caused by focal ischemia such as that inflicted by MCAO. Note that the test is most accurate when large infarcts are caused.

The cage developed for rats by Montoya was redesigned by Campden instruments and Dr. Dunnet for use with mice [Baird A. L. et al., (2001). “—The staircase test of skilled reaching in mice.” Brain Res Bull 54(2): 243-50.] The behavioral test is divided into three parts

a. Training: The animals are trained during a period of a week in the staircase apparatus. At the end of this week the animals are deprived of solid food for the 16 hours leading up to the test. For the test itself, the animals are placed in the apparatus for one hour and the number of pellets collected and knocked down are determined for each paw. At the end of the week the animals reach a performance plateau.

b. Scoring pre-MCAO: The scoring consists of a minimum of three independent measurements. As described above, the animals are deprived of solid food for the 16 hours leading up to the test beginning the night before the experiment. For the test itself, they are placed in the instrument for one hour and the number of pellets collected and knocked down are determined for each paw.

c. Scoring post-MCAO: The animals are allowed to recover from surgery for a week. After the recovery week, at least one measurement, as described above, is performed per week, for a total of four weeks.

All experiments are performed double blind.

For statistical analysis of neurological motor deficiencies measured with the Montoya test, the Wilcoxon signed rank test is used (Siegel, S., Calstellan, N.J. Nonparametric Statistics for the Behavioural Sciences. MacGraw-Hill International Editions, Statistics Series. Second Edition 1988).

3. In situ Determinations

Infarct Size Measurement

(i) Tissue fixation: Animals are sacrificed by decapitation and whole brain is dissected and fixed for 4 hrs in Carnoy's fixative at room temperature. After fixation, samples are washed in three changes of 95% EtOH (20 minutes each wash) and embedded in paraffin by a tissue processor.

(ii) Sectioning: Coronal paraffin sections of brains embedded in paraffin are prepared. Sections are collected and mounted as follows: A series of four sections of 5 μm thickness is cut and two of the slices are mounted on a slide, then 19 sections of 20 μm thickness are cut and discarded. Then, a second series of four 5 μm sections is cut and two of this next set of slices are mounted onto a slide, and so on. This procedure results in a collection of serial thin sections separated by 0.4 mm. Sections collected are used for estimation of infarct volume by stereological Cavalieri's method. [Gundersen, H. J. G. (1988). “Some new, simple and efficient stereological methods and their use in pathological research and diagnosis.” APMIS 96: 379-394; Howard, C. V. and M. G. Reed (1998). Unbiased stereology. Three-dimensional measurement in microscopy., BIOS Scientific Publishers]

(ii) Statistical analysis: For statistical analysis of infarct size the Anova test is used.

All experiments are performed double blind.

Results: The results of in situ analysis are as follows: G-CSF administration may reduce the infarct size.

What is claimed is:

1. A method for promoting recovery in a patient who has suffered a central nervous system injury, the method comprising administering to the patient a colony stimulating growth factor in a dosage sufficient to increase the number of bone-marrow-derived stem cells in the circulation of the patient, so as to thereby promote recovery in the patient.

2. The method of claim 1, wherein the colony stimulating growth factor is selected from a group consisting of granulocyte-colony stimulating factor (G-CSF), granulocyte-macrophage-colony stimulating factor (GM-CSF), stem cell factor (SCF), interleukin-3 (IL-3) and interleukin-6 (IL-6).

3. The method of claim 2, wherein the colony stimulating factor is G-CSF.

4. The method of claim 3, wherein the central nervous system injury comprises an ischemic episode.

5. The method of claim 4, wherein the ischemic episode is stroke.

6. The method of claim 3, wherein the central nervous system injury comprises a traumatic injury.

7. The method of claim 3, wherein administration of the G-CSF begins within one month after the central nervous system injury.

8. The method of claim 7, wherein administration of the G-CSF begins on any one of days 1-30 after the central nervous system injury.

9. The method of claim 8, wherein administration of the G-CSF begins on any one of days 1-7 after the central nervous system injury.

10. The method of claim 5, wherein administration of the G-CSF begins within one month after the stroke.

11. The method of claim 10, wherein administration of the G-CSF begins on any one of days 1-30 after the stroke.

12. The method of claim 11, wherein administration of the G-CSF begins on any one of days 1-7 after the stroke.

13. The method of claim 3, wherein 1 to 1000 microgram (μg) of G-CSF per kg of body weight of the patient is administered one to four times daily for 1 to 14 days.

14. The method of claim 13, wherein 5 to 15 microgram (μg) of G-CSF per kg of body weight of the patient is administered.

15. The method of claim 13, wherein the G-CSF is administered once or twice daily.

16. The method of claim 13, wherein the G-CSF is administered daily for 3 to 5 days.

17. The method of claim 3, wherein administration of G-CSF is effected via intravenous, intraperitoneal, intramuscular or subcutanous injection.
